(19) United States
(12) Patent Application Publication
(10) Pub. No.: US 2022/0272899 A1
(43) Pub. Date: Sep. 1, 2022

(54) APPARATUS FOR HARVESTING MICROALGAE CULTURED TO PRODUCE BIOFUEL AND METHOD OF HARVESTING MICROALGAE USING THE SAME

(71) Applicant: Jae Hoon Kim, Seoul (KR)

(72) Inventor: Jae Hoon Kim, Seoul (KR)

(21) Appl. No.: 17/358,228

(22) Filed: Jun. 25, 2021

(30) Foreign Application Priority Data
Feb. 26, 2021 (KR) 10-2021-0026755

(51) Int. Cl. A01D 44/00; A01G 33/00; C12M 1/00; C12M 1/26; C10L 1/02

(52) U.S. Cl. CPC A01D 44/00; A01G 33/00; C12M 21/02; C12M 21/12; C12M 47/02; C12M 33/16; C12M 29/18; C10L 1/02; C10L 2200/0476; C10L 2200/0484

(57) ABSTRACT

Disclosed herein are a microalgae harvesting apparatus and method. The microalgae harvesting apparatus includes: a gantry crane including traveling towers configured to move along traveling tracks extending along lengthwise directions on both sides of an arrangement of microalgae incubators, a jib installed in the widthwise direction of the traveling towers, a hoist installed on the jib, and a crossbar configured to be selectively lowered and elevated by the hoist; a plurality of harvesters installed to form an N×M array in widthwise and lengthwise directions with respect to the crossbar, and provided with respective doors in the bottom surfaces thereof; a conveyor means configured to move along a lengthwise direction at a height between the microalgae cultivators and the harvesters; and a control panel configured to control the operations of the microalgae harvesting apparatus.

### CROSS-REFERENCE TO RELATED APPLICATION

This application claims the benefit of Korean Patent Application No. 10-2021-0026755 filed on Feb. 26, 2021, which is hereby incorporated by reference herein in its entirety.

### BACKGROUND

### 1. Technical Field

The present invention relates generally to an apparatus for harvesting microalgae cultured to produce biofuel, and more particularly to an apparatus for effectively harvesting microalgae cultured on a large scale.

### 2. Description of the Related Art

Advances across the scientific, medicinal and industrial fields have led to rapid population growth. As the population increases, energy consumption continues to increase. As the use of fossil fuels, which is the basis of energy that supports society today, is rapidly increasing, the imbalance in the supply and demand of energy becomes serious internationally. Since fossil fuels are buried intensively in specific areas, the imbalance in the supply and demand of energy can lead to political and diplomatic conflicts and disputes.

Therefore, interest and efforts in the development of alternative energy are increasing around the world in order to overcome various problems such as the regional concentration of fossil fuels, the depletion of fossil fuels, and increases in the cost of collection of fossil fuels. As a branch, a technology for producing alternative fuels from natural resources that can be obtained in large quantities through cultivation, i.e., a technology for producing biofuel, is attracting attention. Biofuel is a fuel obtained from biomass, and includes not only living organisms but also by-products from metabolic activities such as animal excrement. Biofuel is renewable energy different from fossil fuels, and includes bioethanol and biodiesel.

There are many types of biomass that produce biofuels. Among them, the promising biomass is microalgae. Microalgae are a collective term for a group of organisms having a very small size in the form of single cells that undergo photosynthesis, and have the advantage of being able to grow more efficiently and faster than terrestrial plants. In addition, microalgae have a great advantage in that it is a natural raw material that can minimize the impact on the world's food supply and demand. In other words, biofuels have been mainly produced from corn, soybeans, and sugarcane. When such crops become the main raw materials of biofuels, food shortages may occur due to increased food prices due to fewer grains for people to eat. In contrast, microalgae are biomass that does not cause such concerns.

However, in order for microalgae to be economical as biomass, it is a problem necessary to be solved that it must be cultivated on a large scale. In other words, a facility for cultivating microalgae must be prepared on a large scale of several hundred hectares in order to be competitive with fossil fuels. Furthermore, there is a need to provide a means to effectively harvest microalgae in such a large-scale culture facility.

### RELATED ART DOCUMENT

Patent document: Korean Patent Application Publication No. 10-2020-0108745 (published on Sep. 21, 2020)

### SUMMARY

An object of the present invention is to provide a microalgae harvesting apparatus capable of effectively harvesting microalgae for a large-scale microalgal culture facility.

According to an aspect of the present invention, there is provided a microalgae harvesting apparatus including: a gantry crane including traveling towers configured to move along traveling tracks extending along lengthwise directions on both sides of an arrangement of microalgae incubators, a jib installed in the widthwise direction of the traveling towers, a hoist installed on the jib, and a crossbar configured to be selectively lowered and elevated by the hoist; a plurality of harvesters installed to form an N×M array in widthwise and lengthwise directions with respect to the crossbar, and provided with respective doors in the bottom surfaces thereof; a conveyor means configured to move along a lengthwise direction at a height between the microalgae cultivators and the harvesters; and a control panel configured to control the movement of the gantry crane, the elevation and lowering of the hoist, the opening and closing of the doors, and the movement and transfer of the conveyor means.

The number N of harvesters in the widthwise direction thereof may correspond to the number of microalgae incubators of the arrangement of microalgae incubators in the widthwise direction thereof.

The conveyor means may include a pair of conveyor means in charge of respective halves of the harvesters in the lengthwise direction, and the pair of conveyor means may be merged together or separated from each other by moving in opposite directions with respect to a center in a lengthwise direction thereof.

The pair of conveyor means may perform transfer along the widthwise direction thereof.

Each of the harvesters may be provided with a door opening/closing device having a rod that extends and contracts selectively; and the rod of the door opening/closing device and a corresponding one of the doors may be connected by a link, so that the door is selectively opened and closed by the rod.

The door opening/closing device may be disposed on the top of the harvester connected to the crossbar.

At least a part of sides of the harvester may be formed of a mesh that can discharge water while leaving microalgae.

The gantry crane may include: a nutrient storage tank configured to contain nutrients for the growth of microalgae; and nutrient supply pipes connected to the nutrient storage tank and extended to the microalgae incubators.

According to another aspect of the present invention, there is provided a microalgae harvesting method using the microalgae harvesting apparatus, the microalgae harvesting method including: a first step of moving the conveyor means out of the harvesters; a second step of lowering the hoist to let the harvesters down into the microalgae incubators; a third step of moving the microalgae harvesters out of the microalgae incubators by elevating the hoist after closing the doors of the harvesters; a fourth step of moving the conveyor means under the harvesters, opening the doors of the harvesters, and dropping microalgae onto the conveyor means; and a fifth step of transferring microalgae out of the harvesters by driving the conveyor means.

The second step may include letting the microalgae harvesters down into the microalgae incubators with the doors of the microalgae harvesters opened.

After the fifth step, the gantry crane may be moved to a subsequent column of the arrangement of microalgae incubators, and then the first to fifth steps may be repeated.

### BRIEF DESCRIPTION OF THE DRAWINGS

The above and other objects, features, and advantages of the present invention will be more clearly understood from the following detailed description taken in conjunction with the accompanying drawings, in which:

FIG. 1 is a drawing showing an example of a microalgae incubator arrangement including a plurality of microalgae incubators;

FIG. 2A and FIG. 2B are diagrams showing the overall configuration of a microalgae harvesting apparatus according to the present invention;

FIG. 3A and FIG. 3B are diagrams showing an embodiment of a harvester; and

FIG. 4A, FIG. 4B, FIG. 4C, FIG. 4C, FIG. 4D and FIG. 4E are views illustrating a series of processes for harvesting microalgae using the microalgae harvesting apparatus of FIG. 2A and FIG. 2B.

### DETAILED DESCRIPTION

Embodiments of the present invention will be described in detail below with reference to the accompanying drawings. The advantages and features of the present invention and methods for achieving them will be apparent with reference to the embodiments described below in detail in conjunction with the accompanying drawings. However, the present invention is not limited to the embodiments to be described below, but may be implemented in various different forms. The embodiments are provided merely to make the disclosure of the present invention complete, and to fully convey the scope of the invention to those of ordinary skill in the art. The invention is defined only based on the scope of the claims. The same reference symbols refer to the same components throughout the specification.

Unless otherwise defined, all terms (including technical and scientific terms) used herein may be used in senses that can be commonly understood by those of ordinary skill in the art to which the present invention pertains. In addition, the terms defined in commonly used dictionaries are not interpreted ideally or excessively unless explicitly defined specifically. The terms used herein are intended to describe embodiments, but are not intended to limit the present invention. In this specification, a singular expression also includes a plural expression unless specifically stated in the phrase.

As used herein, “comprises” and/or “comprising” means that recited components, steps, operations and/or elements do not exclude the presence or addition of components, steps, operations, and/or elements.

The present invention will be described in detail below with reference to the accompanying drawings.

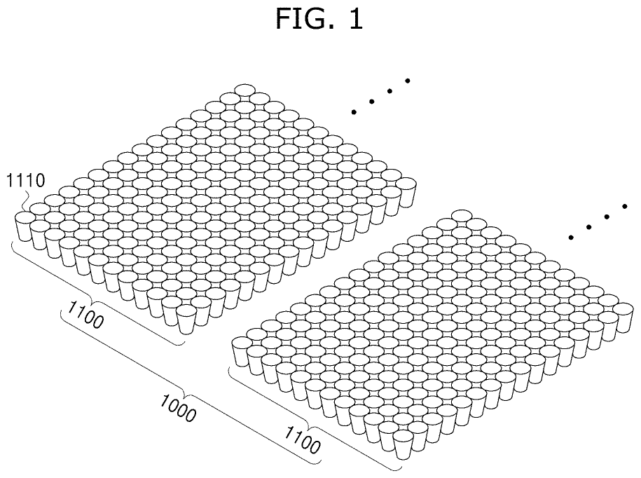

FIG. 1 is a drawing illustrating a microalgae incubator arrangement 1000. FIG. 1 shows an example in which multiple groups 1100 of microalgae incubators in each of which 12 microalgae incubators 1110 are arranged laterally (N in the widthwise direction) and 16 microalgae incubators are arranged vertically are arranged in lateral directions. The size of an actual microalgae incubator arrangement 1000 is considerably larger than this.

In addition, according to the widthwise size of a microalgae harvesting apparatus 10 (hereinafter simply referred to as the “harvesting apparatus”) of the present invention to be described later, separation spaces configured to separate the groups 1100 of microalgae incubators from one another may be formed among the groups 1100 of microalgae incubators in the widthwise direction. The separation spaces in the widthwise direction are provided for traveling tracks 114 for the movement of the gantry crane 100 of the harvesting apparatus 10 and spaces for the collection of microalga.

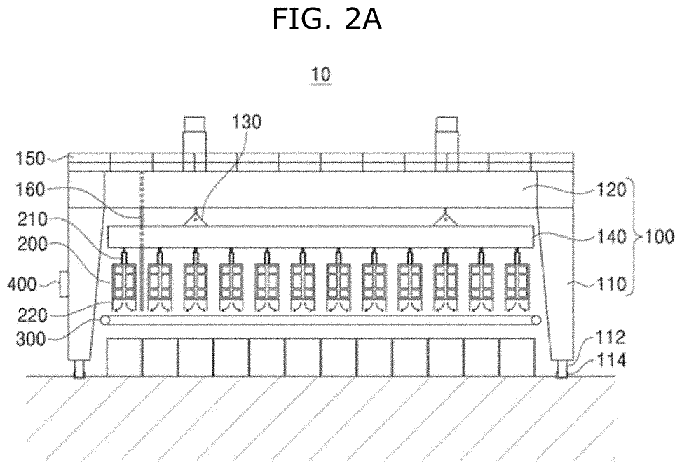

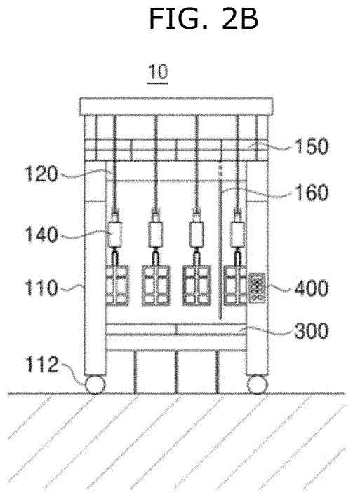

FIG. 2A and FIG. 2B shows the overall configuration of the harvesting apparatus 10 according to the present invention. The shown harvesting apparatus 10 includes the gantry crane 100, a plurality of harvesters 200, a conveyor means 300, and a control panel 400 when the configuration of the harvesting apparatus 10 is basically divided.

The gantry crane 100 forms the whole body of the harvesting apparatus 10, and the harvesters 200, the conveyor means 300, and the control panel 400 are mounted as accessory devices. The gantry crane 100 includes a pair of traveling towers 110 serving as pillars, and the traveling towers 110 move along the traveling tracks 114 extending along the lengthwise directions of both sides of the microalgae incubator arrangement 1000, unlike wheels 112. In addition, a jib 120 serving as a crossbeam is installed in the widthwise direction of the travel towers 110, and hoists 130 are installed on the jib 120. Crossbars 140 disposed in the widthwise direction parallel to the jib 120 are suspended from the hoists 130, and are selectively elevated and lowered according to the operation of the hoist 130. The hoists 130 may include a plurality of hoists by taking into consideration the balance of the crossbars 140 and a load to be suspended.

In addition, the plurality of harvesters 200 is installed to form a N×M array in the widthwise and lengthwise directions with respect to the crossbars 140 of the gantry crane 100. The M crossbars 140 are provided along the lengthwise direction, and harvesters 200 in an N×M array are provided as a whole with N harvesters 200 installed on each of the crossbars 140. In the harvesters 200 shown in FIG. 2A and FIG. 2B, a total of 48 harvesters 200 in a 12×4 arrangement are suspended from the crossbar 140. Each of the harvesters 200 has a cross-sectional size that allows the harvester 200 to be deeply immersed in a corresponding one of the groups 1110 of microalgae incubators, and a door 220 that is selectively opened and closed is provided on the bottom surface of each harvester 200.

In this case, referring to FIG. 1, the number (N) of harvesters 200 in the widthwise direction corresponds to the number of the groups 1100 of microalgae incubators in the widthwise direction (in particular, the number of groups spaced apart from each other in the widthwise direction). The reason for this is that it is efficient to collectively harvest microalgae in the widthwise direction of the microalgae incubator arrangement 1000. All the harvesters 200 are suspended at the same height with respect to the crossbars 140. When there are multiple crossbars 140, it is preferable that all crossbars 140 be selectively elevated and lowered while maintaining the same height.

The gantry crane 100 may travel along the lengthwise direction by the traveling towers 110, and the crossbars 140 may be selectively elevated and lowered by the operation of the hoists 130. Accordingly, all the harvesters 200 suspended from the crossbars 140 may be moved in two directions according to the traveling of the gantry crane 100 and the elevation/lowering of the crossbars 140.

In addition, there is provided a conveyor means configured to move along the lengthwise direction at a height between the microalgae incubators 1110 and the harvesters 200. In other words, the conveyor means moves along the lengthwise direction through a space formed between the harvesters 200 and the groups 110 of microalgae incubators when the harvesters 200 are elevated above the groups 110 of microalgae incubators. Accordingly, the conveyor means 300 may change its position to a position directly under the harvester 200 or to a side outside the harvesters 200.

The conveyor means 300 needs to have a size that covers the overall bottom surface of the plurality of harvesters 200 forming an N×M array. The conveyor means 300 may be provided in the form of a variety of conveyors capable of transporting microalgae such as a belt conveyor or a screw conveyor. In addition, according to an embodiment, the conveyor means 300 may include a pair of conveyor means 300 in charge of respective halves of the harvesters 200 in the lengthwise direction. The pair of conveyor means 300 may be configured to be merged together or separated from each other by moving in opposite directions with respect to with respect to a center in a lengthwise direction thereof. In addition, the pair of conveyor means 300 may collect microalgae along the sides of the traveling tracks 114 by transferring the harvested microalgae along the widthwise direction.

In addition, the harvesting apparatus 10 includes the control panel 400. The control panel 400 controls the traveling of the gantry crane 100, the elevation and lowering of the hoists 130, the opening/closing of the doors 220 of the harvesters 200, and the movement and transfer of the conveyor means. In other words, the control panel 400 serves to perform control so that a series of operations such as the traveling of the gantry crane 100 and the elevation and lowering of the hoists 130 are performed in a predetermined sequence by a preset program or manual operation. A series of operations for the harvest of microalgae will be clearly understood through a description of the harvesting method of the present invention, which will be given later.

In addition, according to an embodiment of the present invention, the gantry crane 100 may include a nutrient storage tank 150 and nutrient supply pipes 160. In FIG. 2A and FIG. 2B, there is shown a nutrient supply pipe 160 for the harvester 200 at the leftmost end. The reason for this is that nutrient supply pipes 160 corresponding to the other harvesters 200 are omitted for the sake of ease of understanding. The nutrient storage tank 150 containing nutrients for the growth of microalgae is disposed on the top of the gantry crane 100, and the nutrient supply pipes 160 directed to the microalgae incubators 1110 are connected to the nutrient storage tank 150. Accordingly, the supply of nutrients to microalgae is automatically performed, thereby enabling mass cultivation. The supply of nutrients to microalgae is also automatically performed by the control panel 400.

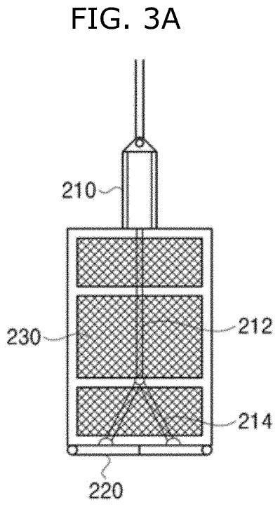

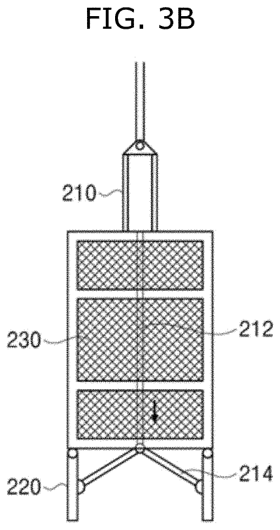

FIG. 3A and FIG. 3B show an embodiment of the harvester 200. Referring to this drawing, the harvester 200 is provided with a door opening/closing device 210 having a rod 212 that extends and contracts selectively. The rod 212 of the door opening/closing device 210 and the door 220 are connected by a link 214. As the rod 212 of the door opening/closing device 210 extends and contracts, the opening and closing operations of the door 220 occur. A hydraulic cylinder or an electric cylinder may be used as the door opening/closing device 210.

At least some of the side surfaces of the harvester 200 are each formed of a mesh 230 capable of discharging water while leaving microalgae. In addition, the door opening/closing device 210 may be disposed on the top of the harvester 200 connected to the crossbar 140. Since water including microalgae is filled into the harvester 200 and then the water is discharged through the mesh 230 on the side, the door opening/closing device 210 is preferably located on the top of the harvester 200 to which water does not come into direct contact with water for the sake of durability and reliability.

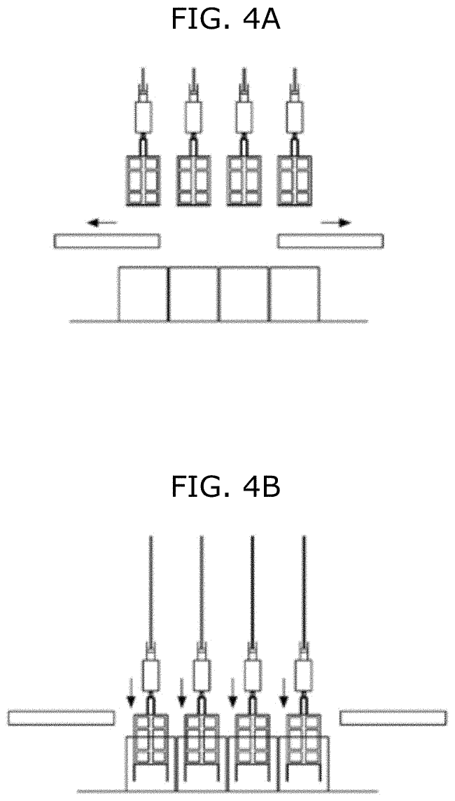

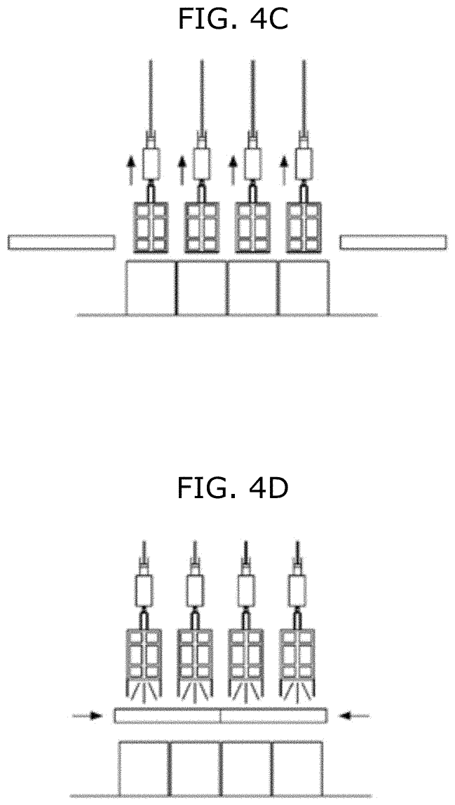

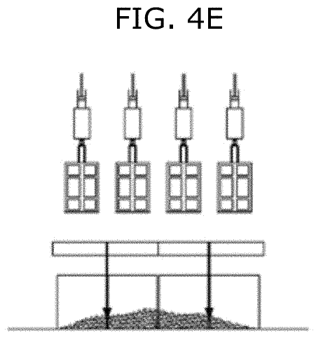

FIG. 4A, FIG. 4B, FIG. 4C, FIG. 4D and FIG. 4E are views illustrating a series of methods or processes for harvesting microalgae using the microalgae harvesting apparatus 10 having the above-described configuration.

FIG. 4A illustrates a first step of moving the conveyor means 300 out of the harvesters 200. The conveyor means 300 moves along the lengthwise direction and empties the space under the harvesters 200, and thus there is provided a passage through which the harvesters 200 can enter the microalgae incubators 1110.

FIG. 4B illustrates a second step of letting the harvesters 200 down into the microalgae incubators 1110 by lowering the hoists 130. The harvesters 200 are filled with water containing a large number of microalgae by sinking the harvesters 200 below the water surfaces of the microalgae incubators 1110. Although the harvesters 200 may be completely sunk to be submerged in the water, it will be more effective to let the harvesters 200 down into the microalgae incubators 1110 with the doors 220 of the harvesters 200 open because the harvesters 200 can be filled with water rapidly.

In addition, FIG. 4C illustrates a third step of moving the harvesters 200 out of the microalgae incubators 1110 by elevating the hoist 130 after closing the doors 220 of the harvesters 200. As the harvesters 200 are moved out of the microalgae incubators 1110, water is discharged through the sides of the harvesters 200, thereby leaving only the collected microalgae in the harvesters 200.

Next, the fourth step shown in FIG. 4D is performed. The fourth step is the step of moving the conveyor means 300, which has been removed to the outside, under the harvesters 200, opening the doors 220 of the harvesters 200, and dropping microalgae onto the conveyor means 300.

In addition, at the fifth step shown in FIG. 4E, the microalgae are transferred out of the harvesters 200 by driving the conveyor means 300. When the conveyor means 300 transfers the microalgae along the width direction, the dropped microalgae are collected on the sides of the traveling tracks 114. When containers such as drums are prepared in accordance with the transfer positions of the conveyor means 300, the microalgae harvested into the containers are collected.

When the first to fifth steps described above are performed, the conveyor means 300 is located under the harvesters 200, and the harvesters 200 return to the initial state of being suspended above it. In a state in which the harvesting apparatus 10 returns to its initial state, the gantry crane 100 may be moved immediately, so the method of harvesting microalgae according to the present invention is significantly efficient in its procedure. In addition, since the harvesting of microalgae of the harvesters 200 and the collection (transfer) of microalgae of the conveyor means 300 are separated from each other, the present invention is more efficient in terms of traffic lines that the case where the harvesters 200 are responsible for both the harvesting and collection of microalgae, and is also advantageous in terms of space efficiency because auxiliary operating spaces (for example, the moving spaces of the harvesters) are required on the sides of the harvesting apparatus 10.

After the performance of the fifth step, the gantry crane 100 is moved to the next column of the microalgal cultivator arrangement 1000, e.g., a fifth column in the longitudinal direction when the width×length arrangement of the harvesters 200 is a 12×4 arrangement, and then repeats the first to fifth steps. When this repeated procedure is performed up to the longitudinal end of the microalgae incubator arrangement 1000, one round of harvesting procedure is completed.

The microalgae harvesting apparatus of the present invention having the above-described configuration enables the effective harvesting of microalgae cultured on a large scale.

In addition, the microalgae harvesting apparatus of the present invention returns to its initial state after going through a series of harvesting processes, so that the movement of the gantry crane for the next harvest is made immediately, with the result that the harvesting procedure is significantly efficient.

Furthermore, since the harvesting of microalgae of the harvesters and the collection of microalgae of the conveyor means are separated from each other, the present invention is more efficient in terms of traffic lines that the case where the harvesters are responsible for both the harvesting and collection of microalgae, and is also advantageous in terms of space efficiency because auxiliary operating spaces are required on the sides of the harvesting apparatus.

While the embodiments of the present invention have been described above with reference to the accompanying drawings, it will be appreciated by those of ordinary skill in the art to which the present invention pertains that the present invention may be implemented in other specific forms without changing the technical spirit or essential features of the present invention. Therefore, it should be understood that the embodiments described above are illustrative and non-limiting in all respects.

What is claimed is:

1. A microalgae harvesting apparatus comprising:
a gantry crane including traveling towers configured to move along traveling tracks extending along lengthwise directions on both sides of an arrangement of microalgae incubators, a jib installed in a widthwise direction of the traveling towers, a hoist installed on the jib, and a crossbar configured to be selectively lowered and elevated by the hoist;
a plurality of harvesters installed to form an N×M array in widthwise and lengthwise directions with respect to the crossbar and provided with respective doors in bottom surfaces thereof;
a conveyor means configured to move along a lengthwise direction at a height between the microalgae cultivators and the harvesters; and
a control panel configured to control movement of the gantry crane, elevation and lowering of the hoist, opening and closing of the doors, and movement and transfer of the conveyor means.

2. The microalgae harvesting apparatus of claim 1, wherein the number N of harvesters in a widthwise direction thereof corresponds to a number of microalgae incubators of the arrangement of microalgae incubators in a widthwise direction thereof.

3. The microalgae harvesting apparatus of claim 2, wherein the conveyor means includes a pair of conveyor means in charge of respective halves of the harvesters in the lengthwise direction, and the pair of conveyor means are merged together or separated from each other by moving in opposite directions with respect to a center in a lengthwise direction thereof.

4. The microalgae harvesting apparatus of claim 3, wherein the pair of conveyor means perform transfer along a widthwise direction thereof.

5. The microalgae harvesting apparatus of claim 1, wherein:
each of the harvesters is provided with a door opening/closing device having a rod that extends and contracts selectively; and
the rod of the door opening/closing device and a corresponding one of the doors are connected by a link, so that the door is selectively opened and closed by the rod.

6. The microalgae harvesting apparatus of claim 5, wherein the door opening/closing device is disposed on a top of the harvester connected to the crossbar.

7. The microalgae harvesting apparatus of claim 1, wherein at least a part of sides of the harvester is formed of a mesh that can discharge water while leaving microalgae.

8. The microalgae harvesting apparatus of claim 1, wherein the gantry crane comprises:
a nutrient storage tank configured to contain nutrients for growth of microalgae; and
nutrient supply pipes connected to the nutrient storage tank and extended to the microalgae incubators.

9. A microalgae harvesting method using the microalgae harvesting apparatus according to claim 1, the microalgae harvesting method comprising:
a first step of moving the conveyor means out of the harvesters;
a second step of lowering the hoist to let the harvesters down into the microalgae incubators;
a third step of moving the microalgae harvesters out of the microalgae incubators by elevating the hoist after closing the doors of the harvesters;
a fourth step of moving the conveyor means under the harvesters, opening the doors of the harvesters, and dropping microalgae onto the conveyor means; and
a fifth step of transferring microalgae out of the harvesters by driving the conveyor means.

10. A microalgae harvesting method using the microalgae harvesting apparatus according to claim 2, the microalgae harvesting method comprising:
a first step of moving the conveyor means out of the harvesters;
a second step of lowering the hoist to let the harvesters down into the microalgae incubators;
a third step of moving the microalgae harvesters out of the microalgae incubators by elevating the hoist after closing the doors of the harvesters;
a fourth step of moving the conveyor means under the harvesters, opening the doors of the harvesters, and dropping microalgae onto the conveyor means; and
a fifth step of transferring microalgae out of the harvesters by driving the conveyor means.

11. A microalgae harvesting method using the microalgae harvesting apparatus according to claim 3, the microalgae harvesting method comprising:
a first step of moving the conveyor means out of the harvesters;
a second step of lowering the hoist to let the harvesters down into the microalgae incubators;
a third step of moving the microalgae harvesters out of the microalgae incubators by elevating the hoist after closing the doors of the harvesters;
a fourth step of moving the conveyor means under the harvesters, opening the doors of the harvesters, and dropping microalgae onto the conveyor means; and
a fifth step of transferring microalgae out of the harvesters by driving the conveyor means.

12. A microalgae harvesting method using the microalgae harvesting apparatus according to claim 4, the microalgae harvesting method comprising:
a first step of moving the conveyor means out of the harvesters;
a second step of lowering the hoist to let the harvesters down into the microalgae incubators;
a third step of moving the microalgae harvesters out of the microalgae incubators by elevating the hoist after closing the doors of the harvesters;
a fourth step of moving the conveyor means under the harvesters, opening the doors of the harvesters, and dropping microalgae onto the conveyor means; and
a fifth step of transferring microalgae out of the harvesters by driving the conveyor means.

13. A microalgae harvesting method using the microalgae harvesting apparatus according to claim 5, the microalgae harvesting method comprising:
a first step of moving the conveyor means out of the harvesters;
a second step of lowering the hoist to let the harvesters down into the microalgae incubators;
a third step of moving the microalgae harvesters out of the microalgae incubators by elevating the hoist after closing the doors of the harvesters;
a fourth step of moving the conveyor means under the harvesters, opening the doors of the harvesters, and dropping microalgae onto the conveyor means; and
a fifth step of transferring microalgae out of the harvesters by driving the conveyor means.

14. A microalgae harvesting method using the microalgae harvesting apparatus according to claim 6, the microalgae harvesting method comprising:
a first step of moving the conveyor means out of the harvesters;
a second step of lowering the hoist to let the harvesters down into the microalgae incubators;
a third step of moving the microalgae harvesters out of the microalgae incubators by elevating the hoist after closing the doors of the harvesters;
a fourth step of moving the conveyor means under the harvesters, opening the doors of the harvesters, and dropping microalgae onto the conveyor means; and
a fifth step of transferring microalgae out of the harvesters by driving the conveyor means.

15. A microalgae harvesting method using the microalgae harvesting apparatus according to claim 7, the microalgae harvesting method comprising:
a first step of moving the conveyor means out of the harvesters;
a second step of lowering the hoist to let the harvesters down into the microalgae incubators;
a third step of moving the microalgae harvesters out of the microalgae incubators by elevating the hoist after closing the doors of the harvesters;
a fourth step of moving the conveyor means under the harvesters, opening the doors of the harvesters, and dropping microalgae onto the conveyor means; and
a fifth step of transferring microalgae out of the harvesters by driving the conveyor means.

16. A microalgae harvesting method using the microalgae harvesting apparatus according to claim 8, the microalgae harvesting method comprising:
a first step of moving the conveyor means out of the harvesters;
a second step of lowering the hoist to let the harvesters down into the microalgae incubators;
a third step of moving the microalgae harvesters out of the microalgae incubators by elevating the hoist after closing the doors of the harvesters;
a fourth step of moving the conveyor means under the harvesters, opening the doors of the harvesters, and dropping microalgae onto the conveyor means; and
a fifth step of transferring microalgae out of the harvesters by driving the conveyor means.

17. The microalgae harvesting method of claim 9, wherein the second step comprises letting the microalgae harvesters down into the microalgae incubators with the doors of the microalgae harvesters opened.

18. The microalgae harvesting method of claim 9, wherein, after the fifth step, the gantry crane is moved to a subsequent column of the arrangement of microalgae incubators, and then the first to fifth steps are repeated.
